(19) United States
(12) Patent Application Publication
(10) Pub. No.: US 2005/0118107 A1
(43) Pub. Date: Jun. 2, 2005

(54) Composition for inhalation

(76) Inventors: Steve Burns, Leicestershire (GB); Philip Wikeley, Worcestershire (GB)

Correspondence Address: FISH & RICHARDSON P.C., 225 FRANKLIN STREET, BOSTON, MA, 02110, US

(21) Appl. No.: 10/503,853

(22) PCT Filed: Jan. 29, 2003
(86) PCT No.: PCT/SE03/00157

(30) Foreign Application Priority Data
Feb. 1, 2002 (SE) 0200412-5
Jul. 5, 2002 (SE) 0202138-4

(51) Int. Cl. A61L 9/04; A61M 11/00

(52) U.S. Cl. 424/45; 128/200.23

(57) ABSTRACT

The invention relats to a novel pharmaceutical composition useful in the treatment of respiratory disorders such as asthma, rhinitis and chronic obstructive pulmonary disease (COPD). The composition comprises formoterol.

### FIELD OF THE INVENTION

The present invention relates to a pMDI formulation of formoterol in a blend of propellants for use in the treatment of inflammatory conditions/disorders, especially respiratory diseases such as asthma, COPD and rhinitis.

### BACKGROUND OF THE INVENTION

Stability is one of the most important factors, which determines whether a compound or a mixture of compounds can be developed into a therapeutically useful pharmaceutical product.

Formoterol is known in the art, and is marketed as Oxis™ in a dry powder inhaler. There are a variety of other inhalers by which a respiratory product can be administered, such as pressurised metered dose inhalers (pMDI's). Formulations for pMDI's may require certain excipients such as those disclosed in WO 93/05765. It is also known that drug deposition can be reduced by internally coating the cans of pMDI's.

It has now been found that certain HFA formulations comprising formoterol together with polyvinylpyrrolidone (PVP) and polyethylene glycol (PEG) exhibit excellent product stability, particularly when contained in pMDI's having internally coated cans and where the pMDI's are wrapped to exclude moisture. The formulations of the invention are stable at ambient temperature for at least 12 months and exhibit good levels of dose uniformity. This is in contrast to an alternative commercial CFC product, which has to be stored in refrigerated conditions prior to dispensing to the patient.

The excipients of the formulation are soluble in the propellant blend, thus overcoming the problems of solubility of PVP in certain propellants such as 134a. An important aspect of the invention is the use of propellant 227 as a solvating agent for PVP.

A major aspect of the invention is the use of the blend to achieve the required levels of PVP K25 for this particular formulation. The result is a physically and chemically stable suspension formulation of superior quality.

### DESCRIPTION OF THE INVENTION

In accordance with the present invention, there is provided a pharmaceutical composition suitable for use in a pMDI having a coated can fitted with a retention valve comprising formoterol, HFA 227, HFA 134a, PVP and PEG.

Preferably the PVP is present from about 0.0001 to about 0.01% w/w and the PEG is is present from about 0.001 to about 0.15% w/w.

Preferably the PVP is present in an amount of 0.001% w/w. Preferably the PVP is PVP K25.

Preferably the PEG is present in an amount of 0.1% w/w. Preferably the PEG is PEG 1000.

The HFA 134a and HFA 227 can be present in any suitable ratio, depending on the level of PVP required. Preferably the HBA227 is present as at least 20% of the propellant mixture. More preferably HFA 134a and HFA 227 are present in a ratio of 75% to 25%.

Preferably the can is coated and fitted with a retention valve. Suitable coatings include PFA, PTFE and FEP polymers, known in the art, which can be applied using known techniques. Alternatively the cans may be coated using plasma techniques.

Suitable retention valves include retention valves such as Valois RCS valves

Preferably the pMDI is packaged in a moisture resistant wrapping such as a foil pouch optionally containing a desiccant.

The compositions of the invention can be inhaled from any suitable MDI device. Doses will be dependent on the severity of the disease and the type of patient, but are preferably below or within the range 2-12 microgram per dose ex actuator, more preferably 4.5 mcg per actuation.

Preferably the concentration of formoterol is such that the formulation delivers formoterol at 4.5 mcg per actuation ex-actuator.

The formoterol can be in the form of a mixture of enantiomers, or as a single enantiomer, e.g. the R,R, S, S, R,S or S,R enantiomer. The formoterol can be in the form of the free base, salt or solvate, or a solvate of a salt, preferably the formoterol is in the form of its fumarate dihydrate salt. Other suitable physiologically salts that can be used include chloride, bromide, sulphate, phosphate, maleate, tartrate, citrate, benzoate, 4-methoxybenzoate, 2- or 4-hydroxybenzoate, 4-chlorobenzoate, p-toluenesulphonate, benzenesulphonate, ascorbate, acetate, succinate, lactate, glutarate, gluconate, tricaballate, hydroxynapaphthalenecarboxylate or oleate.

The pharmaceutical compositions according to the invention can be used for the treatment or prophylaxis of a respiratory disorder, in particular the treatment or prophylaxis of asthma, rhinitis or COPD.

In a further aspect the invention provides a method of treating a respiratory disorder, in particular asthma, rhinitis or COPD, in a marmnal, which comprises administering to a patient a pharmaceutical composition as herein defined.

In an additional aspect the invention provides a pMDI containing a composition as defined above. Preferably the pMDI is packaged in moisture resistant wrapping such as a foil wrap, optionally with desiccant such as silica gel.

Experimental section

The compositions may be produced by cold fill or pressure fill techniques, both techniques. and methods well known in the art. In cold filling, the ingredients are placed in a cooled mixing vessel, cooled liquefied propellant added and a dispersion produced by vigorous stirring. Aliquots of the dispersed composition are then filled into cooled aerosol cans and sealed with a suitable valve, e.g. a metering valve.

In pressure filling, the ingredients are placed in a pressure vessel, liquefied propellant added under pressure through a valve and a dispersion of the ingredients in the liquefied dispersed composition are then filled, under pressure, through the valve into suitable cans provided with appropriate valves, e.g. metering valves.

The following example illustrates the invention:

| Substance | Concentration % w/w |
|---|---|
| PVP K25 | 0.001 |
| PEG 1000 | 0.1 |
| Formoterol | x |
| HFA 227ea | 25 |
| HFA 134a | 75 |

Where x gives a dose of 2-12 micrograms ex actuator
Where PVP = polyvinylpryrrolidone
Where PEG = polyethylene glycol

In order to achieve the required level of 0.001%, the solubility of the PVP had to be determined in both HFA 227ea and HFA 134a.

The level of HFA227 necessary to dissolve the required % w/w of previously specified excipients in the HFA 227/HFA134a blend was determined by the following method:

Method for Solubility

0.005% PVP K25 Solutions

Stock solutions of 0.1% w/wPEG1000 in HFA134a and PEG1000 in HFA227 were prepared in aerosol cans. A series of mixtures containing PVP K25 0.005% w/w were prepared at room temperature, (20 C), using the stock solutions above, resulting in 0.005% w/w PVP K25 in various blend mixtures of 227 and 134a. The PVP had previously been weighed into PET vials that had been crimped with a valve.

The samples were left for 6 hrs to equilibrate.

The clarity of the resulting solution/suspensions was noted. (Results 1, 2, 4 and 5)

0.002% PVP K25

A mix of 0.002% PVP K25 in HFA 227/134a (60:40) was also prepared in the above manner. (Result 3)

For More Dilute Solutions

a) A mixture of 0.02% PVP K25, 0.1% PEG 1000 in HFA 227 was prepared. (Previous work had shown PVP to be soluble at this level in HFA 227 alone)

b) Cans of 0.1% w/w PEG1000 in HFA134a and 0.1%w/w PEG1000 in HFA 227 mixtures) were made up. The temperature was ambient (approx 22° C.).

c) Using the above solutions, mixtures of PVP in varying blends of HFAL 34a and HFA227 were prepared by pressure filling into pre-crimped PET vials. The blends ranged from 5-20% HFA227 w/w. The resulting mixtures were overnight on rollers to equilibrate after which the clarity of the resulting mixture was observed. (Results 6 to 10 and 12)

A final “test” mix of a weighed amount of PVPK25 in a PET precrimped vial was mixed with 100% HFA134a, left for several hours and the clarity noted. (Result 11)

Another control solution of 0.1% PEG1000 in HFA134a and HFA 227 remained clear to throughout.

Data

Data is summarised in tables 1-3.

TABLE 1
Solubility of PVP (as % w/w) in varying propellant ratios

| | HFA134a | | Amount PVP | |
|---|---|---|---|---|
| Result | w/w % | 227 w/w % | (% w/w) | Soluble ? |
| | | 100 | 0.0250 | yes |
| | 50 | 50 | 0.0055 | yes |
| | 60 | 40 | 0.0055 | yes |
| | 60 | 40 | 0.0022 | yes |
| | 65 | 35 | 0.0058 | yes |
| | 70 | 30 | 0.0047 | no |
| | 80 | 20 | 0.0010 | yes |
| | 83 | 17 | 0.0008 | mostly |
| | 88 | 12 | 0.0012 | yes |
| | 91 | 8 | 0.0016 | no |
| | 95 | 5 | 0.001 | cloudy |
| | 100 | 0 | 0.001 | no |
| | 100 | 0 | 0.0008 | no |

A mixture of 0.025% PVP in HFA 227ea remained clear i.e. was soluble
A control is solution of 0.1% w/w PEG 1000 in HFA 134a remained clear i.e. was soluble

Product Performance (Based on Chance in Dose Through Can Life ) of 227 Alone or Blended with HFA 134a Can be Demonstrated Using the Data Below.

Method for determining change in dose is: %=mean value end dose/mean value beginning dose*100

TABLE 2
Change in dose through can life for HFA 227 alone and HFA 227/134a
blend formoterol formulations

| | HFA 227 alone | | | | Blend 227:134a = 75:25 | | | | | |
|---|---|---|---|---|---|---|---|---|---|---|
| | Batch A | | Batch B | | Batch C | | | Batch D | | |
| | Initial | | | | | | | | | |
| Time | 12.3 | | 6.7 | | 7.1/4.6 (dup) | | | 6.5/4.2 (dup) | | |
| point | UW | W | UW | W | UW | W | WD | UW | W | WD |
| 6W | 22.7 | 19.7 | 24.4 | 13.9 | −0.7 | −1 | 9.4 | −3.5 | 0.6 | 8.4 |
| 3M | 33.5 | 26.9 | 30.5 | 27.0 | 8.9 | nd | 3.4 | 9.8 | nd | 10.4 |

| | | | | | |
|---|---|---|---|---|---|
| 9M | Not determined | 15.3 | nd | nd | 26.6 |
| 12M | | 8.2 | nd | nd | 6 |

nd = not done
UW/W = unwrapped/wrapped
WD = wrapped with desiccant

For comparison purposes the data at 30/60 is presented although for the blend product more storage points were used. The 227 only batches were not tested after 3M due to loss of prime problems and the worsening rise in dose through can life.

The data in table 2 indicates that the blend of propellants provides superior dose uniformity even when the cans are stored in an unwrapped state.

TABLE 3
The advantage of using an RCS retention valve, for example,
a Valois DF31, can clearly be seen in the data below, which
shows the dose profile of the product using either RCS
(retention) or ACT (rapid-fill, rapid-drain) valve types.
Valve ACT vs RCS
Dose profile ie rise or fall of dose through can life (shots 1-120)

| | Initial | 6W - UW | 6W - W | 3M - UW | 3M - W |
|---|---|---|---|---|---|
| ACT | 13.4 | 23.1 | 18.9 | nd | 10.9 |
| RCS | −2.8 | −7.24 | 0.49 | −1.17 | −0.21 |

1. A pharmaceutical composition for use in a pMDI having a coated can fitted with a retention valve comprising formoterol, HFA 227, HFA 134a, PVP and PEG.

2. pharmaceutical composition according to claim 1 in which the PVP is present from about 0.0001 to about 0.01% w/w and the PEG is present from about 0.001 to about 0.15% w/w.

3. A pharmaceutical composition according to claim 1 in which the ratio of HFA 134a to HFA227 is 75% to 25%.

4. A pharmaceutical composition according to claim 1 in which the PVP is PVP K25.

5. A pharmaceutical composition according to claim 1 in which the PVP is present in an amount of 0.001% w/w.

6. A pharmaceutical composition according to claim 1 in which the PEG is PEG 1000.

7. A pharmaceutical composition according to claim 1 in which the PEG is present in an amount of 0.1% w/w.

8. A pharmaceutical composition according to claim 1 in which formoterol is in the form of its ftimarate dihydrate salt.

9. A pharmaceutical composition according to any claim 1 for use for the treatment or prophylaxis of a respiratory disorder.

10. A pharmaceutical composition according to claim 1 for use for the treatment or prophylaxis of asthma, rhinitis or COPD.

11. A pMDI containing a composition as defined in claim 1.

12. A pMDI according to claim 11, which is fitted with a retention valve.

13. A pMDI according to claim 11, which is packaged in a moisture resistant wrapping.

14. A pMDI according to claim 13 in which the moisture resistant wrapping is a foil pouch, optionally with desiccant.

15. A method of treating a respiratory disorder in a mammal, which comprises administering to a patient a pharmaceutical composition according to claim 1.
